(19) United States
(12) Patent Application Publication
(10) Pub. No.: US 2012/0251612 A1
(43) Pub. Date: Oct. 4, 2012

(54) Wound Healing Device, Method for Making the Same and Method for Treating a Wound

(76) Inventors: Mohammad Rezaul Karim, Chatkhil (BD); Abdulrahman M. Al-Ahmari, Riyadh (SA); Saeed M. Al-Zahrani, Riyadh (SA); Jeong Hyun Yeum, Daegu (KR); Kil-Soo Kim, Daegu (KR)

(21) Appl. No.: 13/073,339

(22) Filed: Mar. 28, 2011

(51) Int. Cl. A61K 9/70; A61P 17/02; B29B 9/06; A61K 31/575

(52) U.S. Cl. 424/445; 514/182; 264/6

(57) ABSTRACT

A wound healing device includes a mat of aligned nanofibers of polyaniline, o-aminobenzenesulfonic acid copolymer, polyvinyl alcohol and chitsosan oligossacaride. Method for fabricating the mat and treating wounds are also disclosed.

### FIELD OF THE INVENTION

The present invention relates to a wound healing device, method for making a wound healing device and a method for treating a wound and more particularly to an aligned nanofiber mat, a method for making aligned nanofiber mats and methods for treating wounds to provide almost complete healing with an increase in collagen and granulation.

### BACKGROUND OF THE INVENTION

The use of nanofiber matrices for medical applications are well known. For example, a U.S. patent of Laurencin et al., U.S. Pat. No. 6,689,166 discloses hybrid nanofibril matrices for use as tissue engineering devices. As disclosed therein, components in biocompatible scaffolds or matrices of nanometer diameter provide favorable environments for cell adhesion, cell proliferation and directional growth. Fibrous and fibrillar organic and inorganic biocompatible materials of nanometer diameter can be integrated into non-woven three-dimensional matrices conducive for cell seeding and proliferation. These three-dimensional scaffolds or matrices can then be fabricated into appropriate shapes to simulate the hierarchical micro- and macro-geometry of tissues and/or organs to be repaired or replaced.

A U.S. patent of Smith et al., U.S. Pat. No. 6,727,447 discloses nitric oxide-modified linear poly (ethylenimine) fibers and uses thereof. As disclosed therein, a novel coating for medical devices provides nitric oxide delivery using nanofibers of linear poly (ethylenimine) diazeniumdiolate. Linear poly (ethylenimine) diazeniumdiolate releases nitric oxide (NO) in a controlled manner to tissues and organs to aid the healing process and to prevent injury to tissues at risk of injury. Electrospun nanofibers of linear poly (ethylenimine) diazeniumdiolate deliver therapeutic levels of NO to the tissues surrounding a medical device while minimizing the alteration of the properties of the device. A nanofiber coating, because of the small size and large surface area per unit mass of the nanofibers, provides a much larger surface area per unit mass while minimizing changes in other properties of a device.

Finally a U.S. Pat. No. 7,235,295 of Laurencin et al., discloses polymeric nanofibers for tissue engineering and drug delivery. The Laurencin et al. patent discloses polymeric nanofibers which are useful in a variety of medical and other applications, such as filtration devices, medical prosthesis, scaffolds for tissue engineering, wound dressings, controlled drug delivery systems, cosmetic skin masks, and protective clothing. These can be formed of any of a variety of different polymers, either non-degradable or degradable. In a preferred embodiment nanofibers are formed of biodegradable and non biodegradable poly-phosphazenes, their blends with other polyphosphazenes or with organic, inorganic/organometallic polymers as well as composite nanofibers of polyphosphazenes with nanosized particles such as hydroxyapatites.

Notwithstanding the above, it is presently believed that there is a need for an improved wound healing device or mat in accordance with the present invention. There should be a potential commercial market for such devices because they promote almost complete wound healing with increases in collagen and granulation. It is also believed that the devices can be produced at a reasonable cost and can be easily applied to a wound to promote healing thereof.

### BRIEF SUMMARY OF THE INVENTION

In essence the present invention contemplates a wound healing device for promoting enhanced healing of a wound. The device includes a mat of aligned conductive nanofibers of polyaniline and o-aminobenzenesulfonic acid copolymer, polyvinyl alcohol chitosan oligossacaride wherein the fibers have a thickness in the order of 100 nanometers.

A second embodiment of the invention contemplates a method for preparing a nanofiber wound healing mat that includes the steps of: providing a mass of aniline, o-aminobenzenesulfonic acid (PVA) and chitosan oligossacaride (COS). The aniline and o-aminobenzenesulfonic acid is chemically polymerizes using ammonium persulfate as an oxidant while maintaining the oxidant/monomer ratio at 1 to form a PAni-co-PABSA copolymer. In addition, a PVA solution (hereafter referred as S1) is prepared in double distilled water at 80° C. with magnetic stirring for 2 hours. Then the PVA solution is cooled to room temperature and the chitosan oligossacaride powder is dissolved in double distilled water with magnet stirring for 1 hour at room temperature and adding the PVA solution (hereafter referred as S3) and the PAni-co-PABSA copolymer to form PAni-co-PABSA/PVA/COS blend solution and electrospinning the PAni-co-PABSA/PVA/COS blend solution at high voltage power and collect the fibers on electrically grounded aluminum foil to form a nanofiber mat.

Finally, the nanofiber mat is applied to an open wound for a period of up to fifteen days to promote healing.

The invention will now be described in connection with the accompanying drawings.

### DESCRIPTION OF THE DRAWINGS

FIG. 1 shows FE-SEM images of (a) bulk PAni-co-PABSA powder synthesized by in-situ polymerization and (b-d) PAni-co-PABSA/PVA/COS nanofiber mats electrospun from 3 wt. % PAni-co-PABSA containing PVA/COS samples with different magnifications;

FIG. 2 is an XRD spectra of (a) bulk PAni-co-PABSA powder synthesized by in-situ polymerization, and (b-d) electrospun PAni-co-PABSA/PVA/COS nanofiber mats for S2, S4 and S5 (here and afterwards S2, S4 and S5 are referred for PAni-co-PABSA/PVA/COS solutions contained of 1:8:1, 3:16:1 and 1:16:1 ratios, respectively) samples, respectively (PAni-co-PABSA solution concentration=3%, PVA solution concentration=7.5% and COS solution concentration=12.5%);

FIG. 3 is a TGA data of (a) bulk PAni-co-PABSA powder synthesized by in-situ polymerization, (e) as received PVA powder, and (b-d) electrospun PAni-co-PABSA/PVA/COS nanofiber mats with different compositions (PAni-co-PABSA solution concentration=3%, PVA solution concentration=7.5% and COS solution concentration=12.5%);

FIG. 4 is a FT-IR spectra of (a) bulk PAni-co-PABSA powder synthesized by in-situ polymerization, (b) as received PVA powder, and (c-e) electrospun PAni-co-PABSA/PVA/COS nanofiber mats with different compositions (PAni-co-PABSA solution concentration=3%, PVA solution concentration=7.5% and COS solution concentration=12.5%);

FIG. 5 shows the percent of wound areas in rats in groups, post-wounding on days 5, 10 and 15. Values are mean±SEM from 10 animals in each group. * Significantly different from control (p<0.05), # significantly different from Fucidin® treated positive control (p<0.05);

FIG. 6 represents different photos illustrating the gross appearances of wound healing pattern in rats (A) Control, (B) Fucidin® ointment, (C) S1, (D) S2, (E) S3 (F) S4 and (G) S5. Rats received a full-thickness excisional wound on day 0. After wounding, the wounds were treated Doubly distilled water, Fucidin® ointment, S1, S2, S3, S4 and S5;

FIG. 7 shows the histological appearances of wounds from experimental groups on 15 days after wounding of (A) normal (B) control (C) Fucidin® ointment (D) S1, (E) S2, (F) S3, (G) S4 and (H) S5. The skin samples were stained with hematoxylin-eosin; original magnification, ×100. Scales bars, 200 μm. Hematoxylin-eosin staining of skin sections of wound edges from normal, control and material treated rats 15 days after wounding. Arrows mark the hair follicle. ED, epidermis; Hf, Hair follicle; G, granulation tissue; F, fatty tissue; and

FIG. 8 shows the histological appearance of wounds from experimental groups on 15 days healing.

### DESCRIPTION OF THE PREFERRED EMBODIMENTS OF THE INVENTION

Polyaniline, one of the oldest of the conjugated conducting polymers, has always been at the forefront in the search for conducting polymers for commercial applications because of its unique reversible proton dopability, excellent redox recyclability, environmental stability, variable electrical conductivity, which can be ‘tuned’, low cost and easy synthesis. The main drawback of PAni in technological applications is hampered by its poor processability, related to low solubility in common solvents and poor miscibility with other polymers. Both properties are related to the strong interaction between polymer chains, either by coulombic or hydrogen bonding effects. There are several ways to improve processability based on the incorporation of functional groups into the polymer backbone. The added functional groups can decrease the interchain interaction and be able to interact with solvents or other polymers through stronger interactions, such as ion-dipole ones. The incorporation of the functional group can be carried out through post-modification of PAni or by copolymerization of aniline with substituted anilines. The ultimate goal is to control the amount of functional group incorporated per polymer monomeric unit.

Poly (vinyl alcohol) is a water-soluble polymer produced industrially by the saponification of poly(vinyl ester) or poly(vinyl ether), with good chemical and thermal stability. PVA is highly biocompatible and is non-toxic. It can be processed easily and has high water permeability. PVA solutions can form physical gels from various types of solvents. These properties have led to the use of PVA in a wide range of applications in medical, cosmetic, food, pharmaceutical and packaging industries. PVA-containing solutions have been processed by numerous techniques including sol-gel processing, phase separation and freeze-thaw cyclic treatments to produce a variety of structures. Ultrafine PVA fibers, which may have different potential applications, cannot be produced by conventional spinning techniques. Through the processes, such as melt spinning, dry or wet spinning, fibers with diameters ranging from 5-500 nm are generally obtained.

Chitosan is an N-deacetylated derivative of chitin, the second most abundant polysaccharide in nature after cellulose. It is generally regarded as non-toxic, biocompatible and biodegradable. It has many unique functional properties for different applications like high molecular weight, high viscosity, high crystallinity and capacity to hydrogen bond intermolecularly but its insolubility at physiological pH values (7.2-7.4) and the rigid D glucosamine structures lead to the poor solubility of chitosan in common organic solvents as well as in water, restricting the uses, especially in a human body. However, the chitosan depolymerization products, i.e., low molecular weight chitosan (chitosan oligosaccharide), can overcome these limitations and hence find much wider applications in diversified fields.

Electrospinning is a very simple and effective approach to produce nanofibers, including aligned nanofibers and crossbar structures with the diameters ranging from micrometers to a few nanometers scale, which may be attractive for various applications in biomedical engineering, filtration, protective clothing, catalysis reaction.

In a typical electrospinning process, a high voltage is applied to create electrically charged jets of polymer solutions. The jets dry and form nanofibers, which are collected on a target as nonwoven mat. The principle of the electrospinning method is quite simple; the electrostatic field stretches the polymer solution into fibers at the same time as the solvent evaporates. However, the process is difficult to control and several variables have an influence on the properties of the end product. Furthermore, the quality of the fibers is typically inconsistent, for example, the fiber deposition may be uneven or the distribution of fiber diameter may be large.

Wound healing processes can be considered to be a continuous morphological change of cells including the change of cell migration, adherence, etc. Wound healing is a highly developed biological defense mechanism for the prevention of body fluid leakage, protection of the regenerating cellular barrier, and the removal of tissue residues and foreign materials, and can be considered as a short term process inducing tissue regeneration and the removal of tissue debris for wound healing.

In this work, we have demonstrated for the first time, ultrafine PAni-co PABSA/PVA/COS nanofiber mats can be fabricated by using the electrospinning technique and evaluated the effects of PAni-co-PABSA/PVA/COS nanofiber mats on wound healing and wound-size reduction in rats.

### 2. Experimental

### 2.1 Materials

PVA with Pₙ=1700 [hydrolyzed, degree of saponification (DS) 99.9%] was obtained from DC Chemical Co., Seoul, South Korea and COS (Average molecular weight above 10,000; 100% water soluble) was purchased from Kittolife Co., Kyongki-do, Korea and used without further purification. Aniline monomer (99%, Sigma-Aldrich) was distilled under a reduced pressure and kept below 0° C. prior to use. O-aminobenzenesulfonic acid, hydrochloric acid, ammonium per sulfate (APS) and other organic solvents were obtained from Aldrich as reagent grade and were used as received. Fucidin® ointment (Sodium fusidate 20 mg/g, Dongwha Pharmaceutical md. Co, South Korea) was purchased from a local drug store. Doubly distilled water was used as a solvent to prepare all solutions.

### 2.2 Preparation of PAni-co-PABSA

The copolymer (aniline-co-o-aminobenzenesulfonic acid) was synthesized by chemical polymerization of aniline and o-aminobenzenesulfonic acid in an aqueous solution of 1.2M hydrochloric acid using ammonium persulfate as an oxidant in a modified literature procedure. The oxidant/monomer ratio was kept at 1. A typical polymerization of PAni-co-PABSA copolymer was as follows: required amount of Aniline and o-aminobenzenesulfonic acid was dissolved in 200 mL of 1.2M hydrochloric acid aqueous solution. A solution of 4.56 g of ammonium persulfate dissolved in 100 mL of 1.2M hydrochloric acid was then added slowly to the monomer solution with constant stirring at room temperature. After 10-15 min., the colorless solution turned green. The reaction mixture was stirred for an additional 24 h at room temperature, after which the copolymer powder formed was filtered out and washed with a small amount of 1.2M hydrochloric acid and methanol until the filtered solution was colorless. The green powder obtained was dried under vacuum for 48 h.

### 2.3 Preparation of PAni-co-PABSA/P VA/COS Blend Solutions

The PVA solutions (7.5 wt. %) were prepared in doubly distilled water at 80° C. under magnetic stirring for 2 h, and then cooled to room temperature. COS powder was dissolved (12.5 wt. %) also in doubly distilled water under magnetic stirring for 1 h at room temperature. The PAni-co-PABSA/P VA/COS blend solutions were prepared by mixing bulk PAni-co-PABSA (3 wt. %), PVA (7.5 wt. %) and COS (12.5 wt. %) with the contents of 1:8:1, 3:16:1 and 1:16:3 respectively in an aqueous solutions at room temperature and gently stirred for another 2 h.

### 2.4 Electrospinning of PAni-co-PABSA/P VA/COS Nanofiber Mats

During electrospinning, a high voltage power (CHUNGPA EMT Co., Ltd., Seoul, Korea; model CPS-60K02VIT) was applied to the bulk PVA (S1), PVA/COS (S3) and PAni-co-PABSA/PVA/COS solutions contained of 1:8:1, 3:16:1 and 1:16:3 (S2, S4 and S5, respectively) in a syringe via an alligator clip attached to the syringe needle. The applied voltage was adjusted at 5-20 kV. The solution was delivered to the blunt needle tip via syringe pump to control the solution flow rate. Fibers were collected on an electrically grounded aluminum foil placed at 5-20 cm vertical distance to the needle tip.

### 2.5 Animals

48 heads of male Sprague-Dawley rats (6-weeks-old, 160-200 g) from Nara Biotech Co (Pyongtaek, Korea) were used. Animals were maintained in environmentally controlled conditions with a 12-hr light/dark cycle with a temperature of 22±1° C. and relative humidity 50±5%. The animals were fed with a laboratory pellet chow (Purina Korea Inc., Korea) and water adlibitum during the experiment. The rats were acclimatized for 1 week before use. All animal experimental procedures were conducted in accordance with Kyungpook National University Guidelines for the Care and Use of Laboratory Animals.

### 2.6 Wound-Healing Experiments

There were 48 rats in total, which were divided into 8 groups of 6 animals each. Skin wound was induced under ether anesthesia. The dorsum was shaved and sterilized. Two equidistant wounds were made on skin and panniculus camosus muscle of either side of the dorsal middle line using an 8 mm biopsy-punch (Stiefel Laboratory, Germany). Intramuscular injection of 5 mg/kg gentamicin was performed to prevent infection after wounding. Group 1, animals were normal control. Group 2, animals were treated with DW spray on wounds (control). Group 3, animals were treated with Fucidin® ointment as a positive control material. Group 4-8, animals were treated with S1, S2, S3, S4 and S5 on wounds, respectively. All wounds were cleaned daily and PAni-co-PABSA/PVA/COS nanofiber mats, Fucidin®, DW were topically applied to wounds daily after cleaning. All materials were applied evenly in sufficient amounts to cover all wound areas. The rats of each group were scrutinized for 15 d after application, during which the wound surfaces were observed. Wound healing was monitored by taking photographs at the indicated time points. Wound area was calculated for each time point, and wound closure was expressed as percentage of recovery with respect to the initial wound area. The results are shown as relative wound area obtained by the ratio of wound area to the initial wound area. After 5, 10, and 15 days, three rats in each group were sacrificed by cervical dislocation, and skin samples were removed. The central portion of underlying tissue was taken and fixed in 10% neutralized buffered formalin. Each specimen was embedded in a paraffin block and thin sections (4 μm) were prepared, and stained with hematoxylin-eosin, and Masson's trichrome for histological observation.

### 2.7 Statistical Analysis

All data were expressed as the mean±SD. Evaluation of statistical significance was determined by paired and unpaired Student's t-test. p<0.05 was considered significant.

### 3. Results and Discussion

### 3.1. Morphology

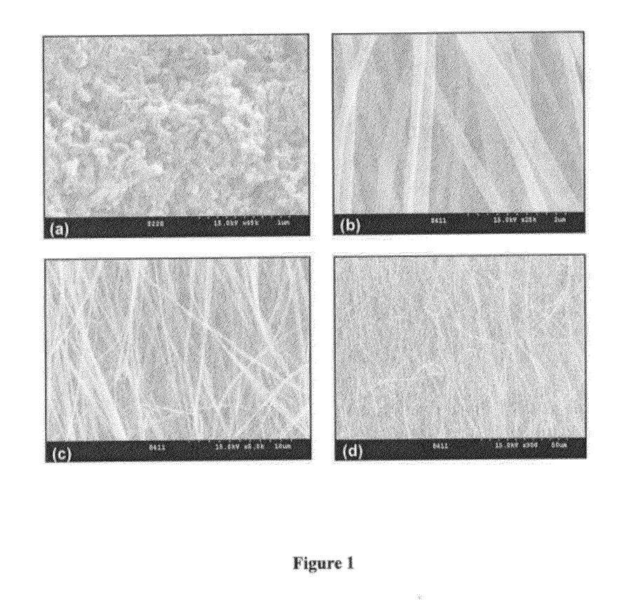

FIG. 1 demonstrates FE-SEM images of bulk PAni-co-PABSA powder by in-situ polymerization and PAni-co-PABSA/PVA/COS nanofiber mats electrospun from 3 wt. % PAni co-PABSA containing PVA/COS samples with different magnifications. The nanofiber mats shown aligned nanofibers by the electrospinning technique.

### 3.2. XRD Data

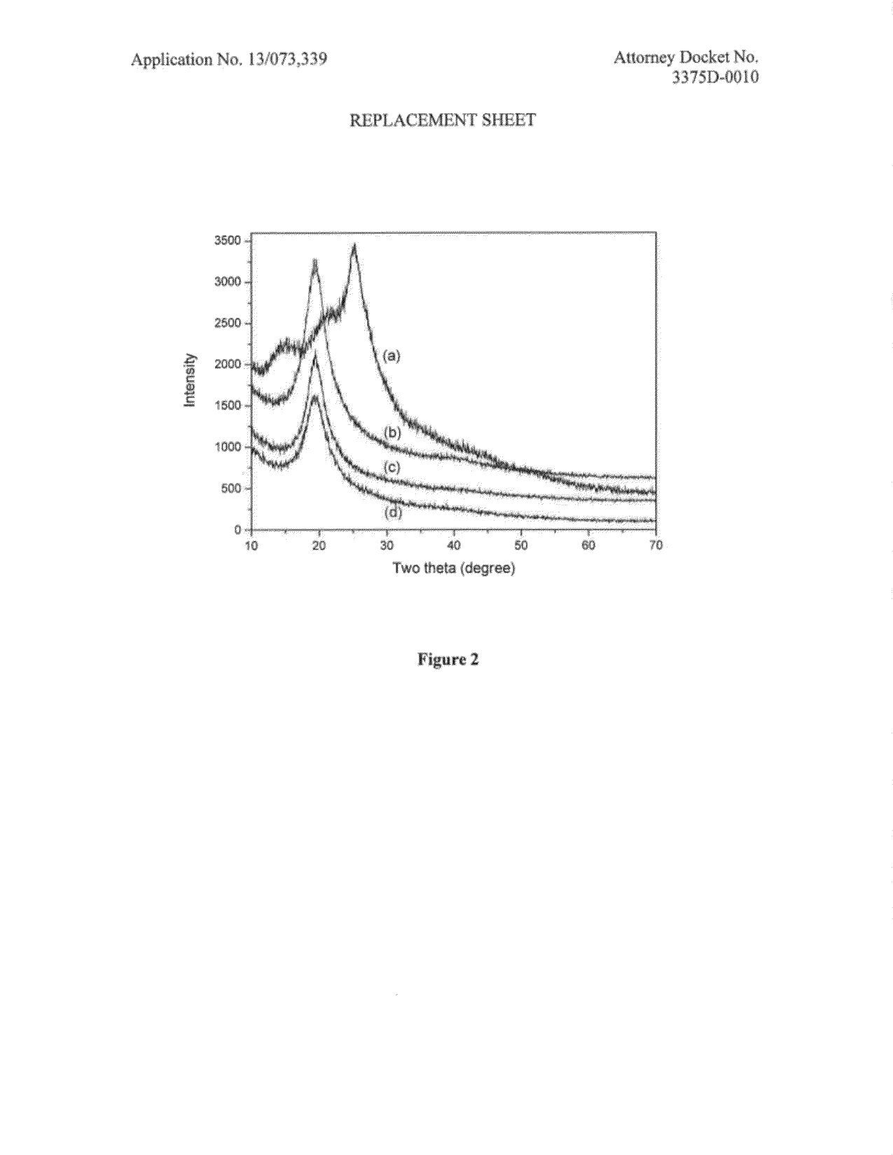

The XRD pattern of bulk PAni-co-PABSA powder by in-situ polymerization and electrospun PAni-co-PABSA/PVA/COS nanofiber mats show in FIG. 2. The bulk PAni-co-PABSA powder shows a significant broad peak at about 25°, which indicates that the copolymers exhibits and amorphous structures (FIG. 2a). This is similar with that of PAni. In case of PAni-co PABSA/PVA/COS nanofiber mats, it shows broad peaks at about 19° (FIG. 2b-d) due to the addition of PVA & COS in the nanofiber mats.

### 3.3. Thermal Stability

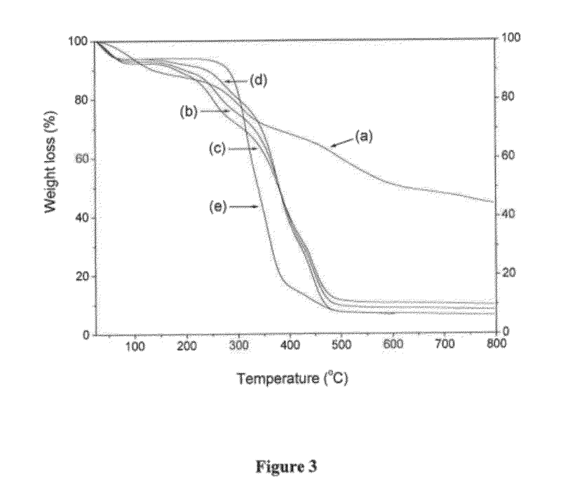

Thermal stability of electrospun PAni-co-PABSA/PVA/COS nanofiber mats is measured using TGA in nitrogen atmosphere. FIG. 3 shows TGA thermograms of different decomposition temperature with bulk PVA, PAni-co-PABSA and PAni-co-PABSA/PVA/COS nanofiber mats. The most below curve of the TGA data [3e] represent the pure PVA and the most upper curve [3a] is for PAni-co-PABSA. FIGS. 3(b-d) are displaying the samples of S2, S4, and S5 at the same trend of thermal stability like the FIG. 3(a, e). Within up to 475° C., there is increased in thermal stability from the bulk PVA nanofibers to PAni-co-PABSA/PVA/COS nanofiber mats. The higher thermal stability might be attributed to its higher contents of PAni-co-PABSA in the PAni-co-PABSA/PVA/COS nanofiber mats.

### 3.5 FT-IR Spectra

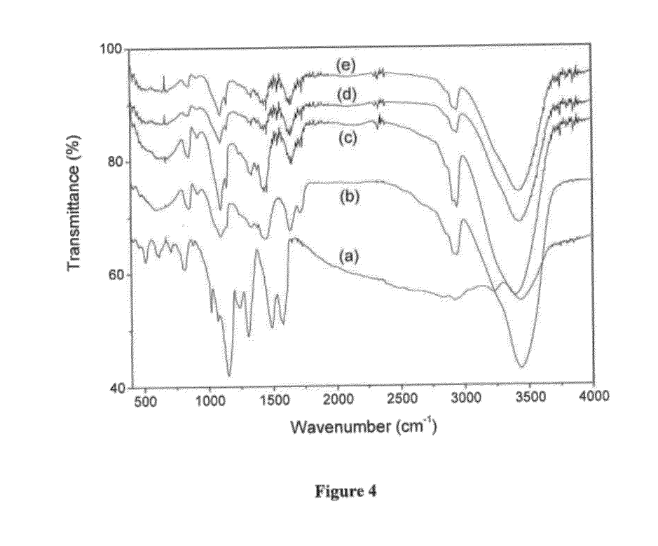

FT-IR spectra give additional information about the structure of nanofiber mats studied. In FIG. 4, examples of spectra of bulk PVA, PAni-co-PABSA and PAni-co-PABSA/PVA/COS nanofiber mats at 400-1000 cm range are shown. Pure PVA exhibits typical bands for vinyl polymers (FIG. 4b). Bands at 2800-3000 cm⁻¹ are due to stretching vibrations of CH and CH groups and bands attributed to CH/CH₂ deformation vibrations are present at 1300-1500 cm⁻¹ range. Also, broad hydroxyl band occurs at 3000-3600 cm⁻¹ and accompanying C-O stretching exists at 1000-1260 cm⁻¹ Low intensive carbonyl band, resulting of residual acetate groups, is detected at 1732 cm⁻¹ in PVA spectrum. The FT-IR spectra of PAni-co-PABSA copolymers display an intense band at 1580 cm which is assigned to the C—C ring stretching vibrations of the benzenoid ring (FIG. 4a). The strong band near 1450 cm is due to C—N stretching mode of the quinoid ring, which arises due to the protonation of polyaniline by the dopant (HCl as well as —COOH of amino benzoic acid). The peak at 1295 cm (strong) corresponds to N—H bending.

The medium intensity band at 1235 cm⁻¹ in the spectra corresponds to C—N stretching modes of the benzenoid ring. A fairly strong band at 935 cm⁻¹ is assignable to the ring-breathing mode of the quinoid group, which becomes active on protonation. The 708 cm⁻¹ band is due to NH wagging of the protonated group. The bands at 1295, 1235 and 708 cm⁻¹ observed in the spectrum of the polyaniline salt remain unshifted in the spectra of the copolymers. The copolymers give rise to bands at around 1690 and 668 cm⁻¹ due to C—O stretching and bending modes, respectively, of amino benzoic acid. In addition, the copolymers show an infrared band around 875 cm⁻¹ which is observed by Thiemann and Brett for the electrochemically synthesized poly (aniline-co-o-amino benzoic acid) copolymer films. The C═C stretching of the benzene ring appears at 1480 cm⁻¹ and the C—N stretching at 1301 cm⁻¹ both are lower than that of bulk PANI. This can be attributed to the lower electron density in the backbone due to the electron-withdrawing capability of the SO₃ group attached directly to the benzene ring. The C—H out-of-plane bending vibrations corresponding to the 1,2,4- and 1,4-substituted benzene rings are at 820 and 870 cm⁻¹ respectively, indicating that the PAni-co-PABSA copolymers have the head-to-tail coupling of the o-aminobenzenesulfonic acid and aniline units.

All these bands are also present in the PAni-co-PABSA/PVA/COS nanofiber mats (FIGS. 4c-e). Intensities of some absorption peaks alter or disappear due to add of PAni-co-PABSA copolymer with PVA and COS. This suggests that hydrogen bonds between hydroxyl groups in PVA and amino groups in copolymers and/or hydroxyl groups in COS could possibly play a role in the shift of the peaks. Therefore, the addition of PVA could moderate the interaction between COS macromolecules and PAni-co-PABSA copolymers, and thus improve the electrospinnability of PAni-co-PABSA and COS with PVA.

### 3.6 Wound Healing Appraisals

The present study is undertaken to evaluate the potentials for use of PAni-co PABSA/PVA/COS nanofibers in wound healing in rats. PAni-co-PABSA/PVA/COS nanofibers show toxicity in rats throughout the experimental period. There is no disorder of the skin in PAni-co-PABSA/PVA/COS nanofibers-treated wounds as compared to control wounds. These results indicate that PAni-co-PABSA/PVA/COS nanofibers are bio-safe and biocompatible.

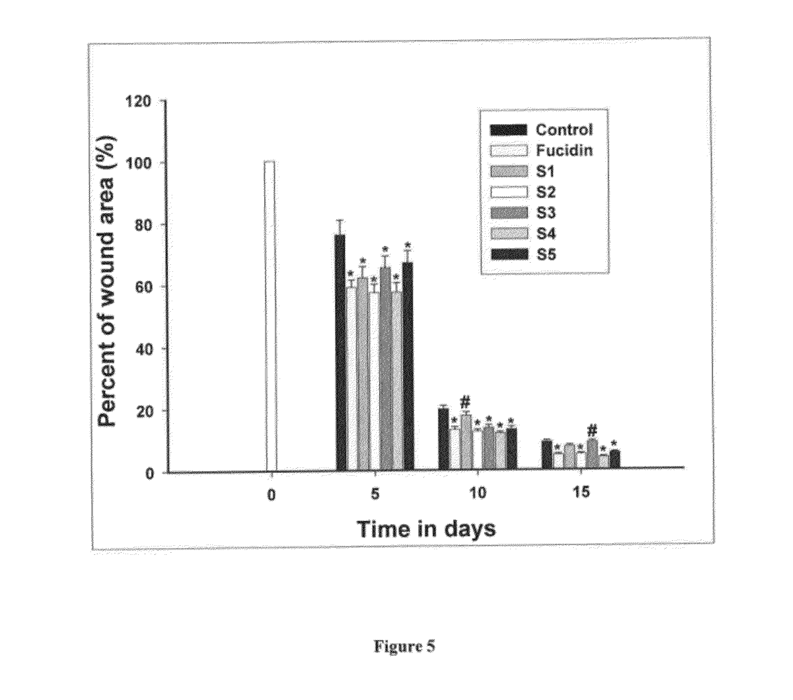

FIG. 5 shows changes of wound area from rats in experimental groups on 0, 5, 10, 15 days after wounding. All wound area measurements are expressed as percentages of initial wound size. The wound area of PAni-co-PABSA/PVA/COS nanofiber treated rats is decreased noticeably in comparison with control rats from 5 days of wounding, and on the day 15. The wound areas of S2, S4 and S5 treated rats are very significant small size wounds as less than 5% of initial wound area. However, the trend of wound healing from the 5 days to the 15 days is a little weak in control rats. These results indicate that PAni-co-PABSA/PVA/COS nanofiber and Fucidin® ointment treated wounds show a statistically significant decrease in percent of wound area as compared to control wounds (p<0.01). This might have been due to the wound healing properties of PAni-co-PABSA/PVA/COS nanofiber and Fucidin® ointment.

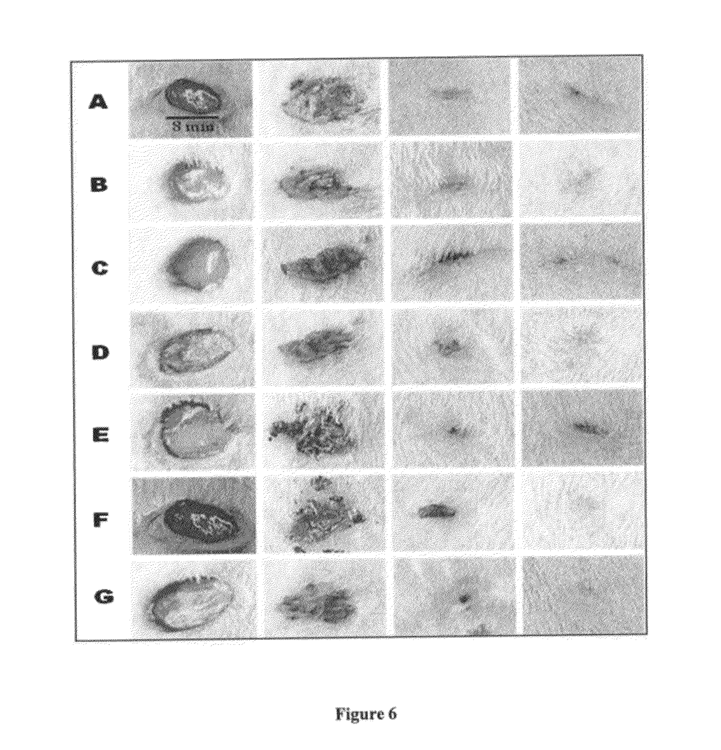

Gross appearances of wounds in experimental groups on 0, 5, 10, and 15 days after treatment with PAni-co-PABSA/PVA/COS nanofiber are illustrated in FIG. 6. The inflammatory reactions in PAni-co-PABSA/PVA/COS nanofiber treated wounds (FIG. 6C-G) tend to be much smaller than those in the control wound (FIG. 6A). During the first few days after wounding, there are inflammations of wounds in all rats, but the wounds improve substantially from 5 days after PAni-co-PABSA/PVA/COS nanofiber and Fucidin® treatment. In PAni-co-PABSA/PVA/COS nanofiber treated rats, wound closure is markedly progressed from the 5 days; however, it is retarded in control rats (FIG. 6A). Although healing of the remainder of the wound to completion is generally variable and dependent upon other factors, regeneration of epithelial cells in PAni co-PABSA/PVA/COS nanofiber treated skins are progressed noticeably before the 5 days.

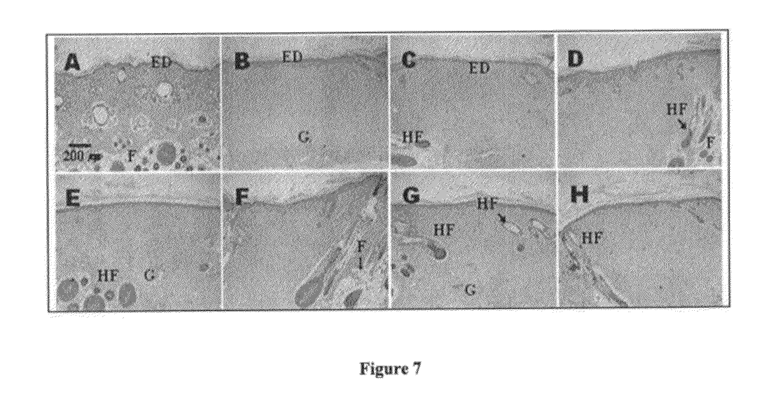

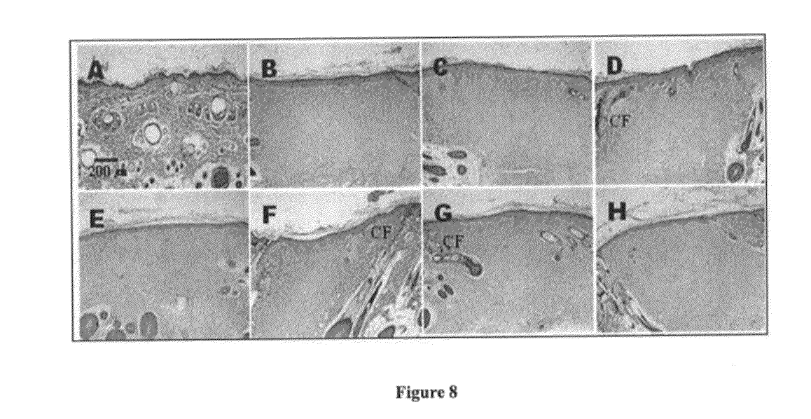

Histological results of wounds from experimental groups on 15 days after treatment with PAni-co-PABSA/PVA/COS nanofiber are shown in FIGS. 7 and 8. The skin samples are stained with hematoxylin-eosin (FIG. 7) and Masson's trichrome (FIG. 8). As illustrated in FIG. 7, the control wounds are not fully epithelialized, and some inflammations are present (FIG. 7B). In control wounds, unevenness of epidermis, decrease of collagen, and increase of inflammation are observed (FIG. 7B) compared with the PAni-co-PABSA/PVA/COS nanofiber-treated wound (FIGS. 7E, 7G, and 7H).

Although epithelialization is observed over the all wounded area, wounds treated with Fucidin® ointment, S2, S4, and S5 are fully covered by an intact epithelium (FIG. 7C, 7E, 70, 7H). The S2, S4, and S5-treated wounds show good healing property, while the S1 and S3-treated wounds displayed incomplete healing (FIG. 7D, 7F). These results indicate that PAni-co PABSA/PVA/COS nanofiber is able to enhance the rate of epithelialization in wound. Masson's trichrome stain of healed scars, which stains blue on collagen fibers as a major component of connective tissue, while the red color represents cytoplasm, red blood cells, and muscle, shows dense collagen in PAni-co-PABSA/PVA/COS nanofiber-treated wounds (FIG. 7D, 7E, 7F, 70, 7H). Collagen is the most common protein in animals and ultimately provides the tensile strength of healing in wounds. The pattern of staining intensity corresponds to the relative quantity of collagen-fiber deposit, which reflexes the process of synthesis and degradation and remodeling as well as the timing of the wound lesion. The wounds treated with PAni-co-PABSA/PVA/COS nanofiber show complete healing on 15 days of treatment and surface of epidermis became even (FIGS. 7E, 7G, and 7H). These results represent that PAni-co-PABSA/PVA/COS nanofiber promote epidermis growth, as shown by full recovery of the epidermis to its normal thickness in PAni-co-PABSA/PVA/COS nanofiber-treated wounds and complete healing and increase in collagen in wounds. Based on the result of the histological studies, it could be confirmed that wound healing is markedly more rapidly progressed in PAni-co-PABSA/PVA/COS nanofiber treated wounds.

### 4. Conclusions

Conducting polyaniline copolymer (PAni-co-PABSA)/poly (vinyl alcohol) (PVA)/chitosan oligossacaride (COS) nanofibers have been fabricated by the electrospinning technique. Firstly, polyaniline copolymer is synthesized by the in-situ polymerization method to make PAni derivatives as a soluble polymer. Secondly, PVA and COS polymers are incorporated with PAni copolymer in an electrospinning technique for fabricating aligned nanofiber mats. The PAni-co PABSA/PVA/COS nanofibers are assembled for wound healing using SD rats by generating two full-thickness skin wounds on the dorsum. PAni-co-PABSA/PVA/COS nanofibers treated wounds have much smaller inflammatory reactions throughout the experimental period. Area of PAni-co-PABSA/PVA/COS nanofibers treated wounds is significantly decreased compare to control and commercially (Fucidin®) ointment-treated wounds. Histological appearance after 15 days of treatment with PAni-co-PABSA/PVA/COS nanofiber reveal almost complete healing and an increase in collagen and granulation as compare to control.

While the invention has been described in connection with its preferred embodiments it should be recognized that changes and modifications may be made therein without departing from the scope of the appended claims.

What is claimed is:

1. A wound healing device for promoting enhanced healing of a wound comprises:
a mat of aligned conductive nanofibers polyaniline and o-aminobenzenesulfonic acid copolymer, vinyl alcohol and chitosan oligossacaride.

2. A wound healing device for promoting enhanced healing of a wound according to claim 1 in which said nanofibers have a thickness in the order of 100 nanometers.

3. A wound healing device for promoting enhanced healing of a wound according to claim 2 in which said nanofibers have an average thickness of between 100 and 1,000 nm.

4. A wound healing device for promoting enhanced healing of a wound according to claim 3 in which said nanofibers have been fabricated by an electro spinning technique.

5. A method for preparing a nanofiber wound healing device comprising the steps of
providing a mass of aniline, o-aminobenzenesulfonic acid PVA and chitosan oligossacaride (COS);
chemically perimerizing the aniline and o-aminobenzenesulfonic acid using ammonium persulfate as an oxidant and maintaining the oxicant/monomer ratio at 1 to form a PAni-co-PABSA copolymer;
preparing a PVA solution in double distilled water at 80° C. under magnetic stirring for 2 hours;
cooling the PVA solution to room temperature;
dissolving chitosan oligossacaride powder in double distilled water under magnetic stirring for 1 hour at room temperature and adding the PVA solution and the PAni-co-PABSA copolymer to form PAni-co-PABSA/PVA/COS blend solution; and
electro spinning the PAni-co-PABSA/PVA/COS blend solution at high voltage power and collect fibers on an electrically grounded aluminum foil to form a nanofiber mat.

6. A method for preparing a nonofiber would healing device according to claim 5 in which the PAni-co-PABSA/PVA/COS blend solution is from a needle tip and electrospinning at an applied voltage of 5-20 KV and whereas fibers were fabricated into aligned nanofiber mats on an electrically grounded aluminum foil placed at 5-20 cm vertical distance from the needle tip.

7. A method for preparing a nanofiber wound healing device according to claim 6 in which the electrospinning high voltage creates electrically charged jets of polymer solution that dry and form nanofibers that are collected on a target as a non-woven mat.

8. A method for preparing a wound healing and wound size reduction consisting of the following steps:
preparing a copolymer of aniline, o-aminobenzenesulfonic acid and chemically polymerizing in aniline and o-aminobenzenesulfonic acid in an aqueous solution of 1.2M HCl using ammonium persulfate as an oxidant;
maintaining the oxidant/monomer ration of 1;
dissolving the aniline and o-aminobenzenesulfonic acid in 200 ml of 1.2 M HCL solution to produce a monomer solution;
dissolving 4.56 g of ammonium persulfate in 100 ml of 1.2 M HCL and then adding slowly to the monomer solution with constant stirring at room temperature for 24 hours to form a copolymer powder;
filtering out the copolymer powder and washing with a small amount of 1.2 M HCL until the filtered solution is colorless to thereby obtain a green powder;
drying the green powder under a vacuum for 48 hours;
preparing a 7.5 wgt. % PVA solution in double distilled water at 80° C. with magnetic stirring for 2 hours and cooling to room temperature;
dissolving 12.5 wgt. % of COS powder in double distilled water with magnetic stirring for 1 hours at room temperature;
preparing a PAni-co-PABSA/PVA/Cos blend solution by mixing bulk PAni-co-PABSA (3 wgt. %), PVA (7.5 wgt. %) and COS (12.5 wgt. %) with the content of between 1:8:1 to 1:16:3 in aqueous solution at room temperature with gentle stirring for 2 hours; and
electrospinning the PAni-co-PABSA/PVA/COS solution at 5-20 KV from a blunt needle tip via a syringe pump to control the solution flow rate and collecting fibers on an electrically grounded aluminum foil placed at 5-20 cm vertical distance from the needle tip.

9. A method for treating a wound by applying an aligned nanofiber mat of PAni-co-PABSA/PVA/COS nanofiber to the wound for a period of 15 days.
